(19) United States
(12) Patent Application Publication
(10) Pub. No.: US 2023/0126643 A1
(43) Pub. Date: Apr. 27, 2023

(54) CELL CULTURE CONTAINER

(71) Applicant: Oribiotech LTD, London, Greater London (GB)

(72) Inventors: Farlan Veraitch, London, Greater London (GB); Jason Palmer, Yaxley Cambridgeshire (GB); Arman Amini, London, Greater London (GB); William Raimes, London, Greater London (GB)

(21) Appl. No.: 17/906,019

(22) PCT Filed: Mar. 9, 2021
(86) PCT No.: PCT/GB2021/050580

§ 371 Date: Sep. 9, 2022

(30) Foreign Application Priority Data
Mar. 9, 2020 (GB) 2003403.9
Mar. 9, 2020 (GB) 2003406.2
Dec. 16, 2020 (GB) 2019859.4

(51) Int. Cl. C12M 1/00; C12M 3/00; C12M 1/04; C12M 1/06

(52) U.S. Cl. CPC C12M 23/26; C12M 23/46; C12M 23/24; C12M 23/22; C12M 23/20; C12M 27/02

(57) ABSTRACT

A cell culture container comprising a base section and a compressible wall element. The base section includes a substantially planar rigid base plate. The compressible wall element extends from the base section in an axial direction and defines an internal volume of the cell culture container. The compressible wall element is compressible in the axial direction. There is also disclosed a bioreactor that includes the cell culture container.

### CROSS-REFERENCE TO RELATED APPLICATIONS

This application is a national phase entry under 35 U.S.C. § 371 of International Patent Application PCT/GB2021/050580, entitled “CELL CULTURE CONTAINER,” filed Mar. 9, 2021, designating the United States of America and published as International Patent Publication WO 2021/181080 A1 on Sep. 16, 2021, which claims the benefit under Article 8 of the Patent Cooperation Treaty to Great Britain Patent Application Serial Nos. 2003406.2 and 2003403.9 filed Mar. 9, 2020, and Great Britain Patent Application Serial No. 2019859.4 filed Dec. 16, 2020.

### TECHNICAL FIELD

The present disclosure relates to a cell culture container, for example, a cell culture container of a bioreactor for use in a cell culturing process.

### BACKGROUND

Cell and gene therapy manufacturing processes are often complex and include manual or semi-automated steps across several devices. Equipment systems used in various steps (i.e., unit operations) of cell-based therapeutic products (CTP) manufacturing may include devices for cell collection, cell isolation/selection, cell expansion, cell washing and volume reduction, cell storage and transportation. The unit operations can vary immensely based on the manufacturing model (i.e., autologous versus allogenic), cell type, intended purpose, among other factors. In addition, cells are “living” entities sensitive to even the simplest manipulations (such as differences in a cell transferring procedure). The role of cell manufacturing equipment in ensuring scalability and reproducibility is an important factor for cell and gene therapy manufacturing.

In addition, cell-based therapeutic products (CTP) have gained significant momentum thus there is a need for improved cell manufacturing equipment for various cell manufacturing procedures, for example, but not limited to, stem cell enrichment, generation of chimeric antigen receptor (CAR) T cells, and various cell manufacturing processes such as collection, purification, gene modification, incubation/recovery, washing, infusion into patient and/or freezing.

The culture or processing of cells typically requires the use of a device to hold the cells, for example, in an appropriate culture medium when culturing the cells. The known devices include shaker flasks, roller bottles, T-flasks and bags.

A key limiting factor in the production of cells or gene therapies for use in medicine is the absence of compact, automated closed systems for performing unit operations without contamination. For example, during cell culture, upstream or subsequent processing of cells, there is a risk of contamination when making additions to the culture vessel, or when removing cells or removing liquid samples. The operating systems are largely manual and hence expensive to operate. Multiple pieces of equipment are typically required to cover all of the non-cell culture steps, which involve many transfers, each of which is an opportunity for operator errors and contamination to occur. Furthermore, with increasing manual operations comes increasing risk of manual errors and, therefore, the current labor-intensive processes lack the robustness required for the manufacture of clinical-grade therapeutics.

There is, therefore, a need for cell processing devices (e.g., multistep cell processors) that permit such processing that avoids the requirement for constant movement of cells into fresh devices.

### BRIEF SUMMARY

According to an aspect of the present disclosure, there is provided a cell culture container comprising a base section and a compressible wall element. The compressible wall element extends from the base section in an axial direction and defines an internal volume of the cell culture container. The compressible wall element is compressible in the axial direction. The base section comprises a substantially planar rigid base plate.

The planar rigid base plate provides a substantially planar, i.e., flat, bottom of an internal volume of the cell culture container. Advantageously, a planar bottom of the cell culture container may provide improved cell culturing, for example, improved mixing and control over cell culturing. The planar bottom of the cell culture container may help to ensure that cells are substantially evenly spread over the cross-section of the cell culture container as the cells will sink to the bottom of the cell culture container. In contrast, if the base section were not planar the cells would be concentrated in a smaller volume, which may be detrimental to cell culturing. The planar rigid base plate of the cell culture container also helps to prevent fluid being trapped in the cell culture container when the cells are harvested or extracted at the end of the cell culturing process.

In some examples, the compressible wall element may be adhered or welded to the rigid base plate. For example, the compressible wall element may be hot plate welded, or ultrasonically welded, to the rigid base plate. In other examples, the compressible wall element may be clamped or clipped onto the rigid base plate. For example, the compressible wall element may comprise a flange that is clipped into a groove formed on the rigid base plate. In some examples, the compressible wall element may be integrally molded with the rigid base plate. For example, the rigid base plate may be over-molded onto a part of the compressible wall element. In particular, the rigid base plate may be over-molded onto a skirt of the compressible wall element. In some examples, the compressible wall element is sealingly attached to the rigid base plate.

In some examples, the compressible wall element may comprise a deformable portion disposed at or immediately adjacent to a joint between the compressible wall element and the rigid base plate. Such an arrangement ensures that the compressible wall element can be completely compressed, and prevents or reduces fatigue stress in the compressible wall.

In other examples, the cell culture container may further comprise a base sheet extending over the rigid base plate within the internal volume of the cell culture container. The base sheet may define a bottom surface of the internal volume of the cell culture container. The base sheet may extend from the compressible wall element. In particular, the base sheet may be integrally molded with the compressible wall element, for example, the compressible wall element and the base sheet may be integrally formed by blow molding. Alternatively, the base sheet may be attached to the compressible wall element and/or the rigid base plate, for example, by adhesive or welding.

In some examples, the base sheet maybe gas permeable. In particular, the base sheet may be oxygen permeable. In other examples, the base sheet may comprise a silicone.

In other examples, the rigid base plate may comprise one or more gas permeable openings. The one or more gas permeable openings may each comprise a hole or aperture in the rigid base plate. Accordingly, gas flow, for example, air flow, is provided to the outer surface of the base sheet through the one or more openings in the rigid base plate.

In some examples, the rigid base plate may comprise one or more spacers adapted to space the base sheet from the rigid base plate. Accordingly, gas flow, for example, air flow, can be provided to a greater surface area of the outer surface of the base sheet.

In other examples, the compressible wall element may comprise an inwardly deformable portion, an outwardly deformable portion, and a leaf portion extending between the inwardly deformable portion and the outwardly deformable portion. In this arrangement deformation of the inwardly deformable portion and the outwardly deformable portion causes the compressible wall element to compress. In particular, the leaf portions can fold against each other to reduce a height of the compressible wall element in the axial direction. It will be appreciated that by the same or similar mechanism the compressible wall element is also extendible.

In some examples, one of the inwardly deformable portion or the outwardly deformable portion is disposed at or immediately adjacent to a joint between the compressible wall element and the rigid base plate. Such an arrangement ensures that the compressible wall element can be completely compressed, and prevents or reduces fatigue stress in the compressible wall.

In other examples, the rigid base plate may comprise a transparent or translucent sensor window. In some examples, the rigid base plate is transparent or translucent, and a portion of the rigid base plate comprises the sensor window. In other examples, the rigid base plate is opaque and the rigid base plate comprises an attached, inserted, or integrally molded transparent or translucent sensor window. In some examples, the rigid base plate comprises an opaque high density polyethylene and a transparent polycarbonate sensor window attached to or integrally molded with the rigid base plate. In other examples where the cell culture container comprises a base sheet, the base sheet may comprise a transparent or translucent material. Alternatively, the base sheet may comprise an opening corresponding to the sensor window. In this example, the base sheet may be sealed to the rigid base plate about the opening.

In some examples, the cell culture container comprises one or more sensor elements disposed on the sensor window. The one or more sensor elements may be disposed on an internal surface of the sensor window, within the internal volume of the cell culture container. The one or more sensor elements may comprise an optical dot, for example, an optical dot for use with an optical fluorescence sensor.

In other examples, the cell culture container may further comprise one or more sensors, in particular, optical sensors, mounted at the sensor window. The one or more optical sensors may transmit and receive light through the sensor window to detect a parameter of the fluid within the cell culture container. The one or more optical sensors may cooperate with the one or more sensor elements, in particular, optical dots. The optical sensors and/or optical dots may measure a dissolved oxygen concentration of the fluid within the cell culture container.

In some examples, the compressible wall element may comprise a silicone. In other examples, the compressible wall element may comprise a low density polyethylene. In other examples, the compressible wall element may comprise a thermoplastic elastomer. In other examples, the compressible wall element may comprise an outer portion and a liner or insert. For example, the outer portion may comprise a thermoplastic elastomer, and the liner may be blow-molded into the outer portion. In another example, the compressible wall element may comprise an inner portion and a jacket. The jacket may be over-molded onto the inner portion. In some examples, at least a part of the compressible wall element may comprise a coating, for example, a gas impermeable coating. In some examples, at least a part of the compressible wall element comprises a gas impermeable coating.

In other examples, the rigid base plate may comprise a high density polyethylene or a polycarbonate. In some examples, the rigid base plate is transparent or translucent, or comprises a transparent or translucent sensor window.

According to a further aspect of the present disclosure, there is provided a bioreactor for a cell culturing process. The bioreactor comprises the cell culture container described above. The bioreactor may further comprise an interface plate attachable to the compressible wall element opposite to the base section to close the cell culture container. The interface plate may act as a lid or closure for the cell culture container. The interface plate may seal the internal volume of the cell culture container.

In some examples, the interface plate may comprise a connector interface. For example, the connector interface may facilitate fluid connection of a container for inputting a fluid into the cell culture container, or fluid connection of a sampling vessel for sampling a fluid in the cell culture container.

In other examples, the bioreactor may further comprise one or more sensors, in particular, one or more optical sensors, arranged at a sensor window in the rigid base plate. The one or more optical sensors may transmit and receive light through the sensor window to detect a parameter of the fluid within the cell culture container. The one or more optical sensors may cooperate with one or more sensor elements, in particular, optical dots, disposed on the sensor window. The optical sensors and/or optical dots may measure a dissolved oxygen concentration of the fluid within the cell culture container.

According to a further aspect of the present disclosure, there is provided a cell processing system comprising the bioreactor as described above.

In some examples, the cell processing system may further comprise an agitator configured to move the base section to agitate a fluid in the cell culture container. The agitator may be configured to move the base section relative to the interface plate by at least partially compressing or extending the compressible wall.

### BRIEF DESCRIPTION OF THE DRAWINGS

Embodiments of the disclosure are further described hereinafter with reference to the accompanying drawings, in which:

FIG. 1 shows a bioreactor having a cell culture container;

FIG. 2A shows a cross-section of an example cell culture container;

FIG. 2B shows an exploded assembly view of the cell culture container of FIG. 2A;

FIG. 3A shows an example cell culture container;

FIG. 3B shows a cross-section of the cell culture container of FIG. 3A;

FIGS. 3C and 3D show detailed cross-sections of example joints between the compressible wall element and rigid base plate of the cell culture container of FIGS. 3A and 3B;

FIG. 4 shows a cross-section of an example cell culture container;

FIGS. 5A to 5C show an example of a cell culture container;

FIG. 6A shows a cross-section of an example cell culture container;

FIG. 6B shows an exploded assembly view of the cell culture container of FIG. 6A;

FIG. 7A shows a cross-section of an example cell culture container;

FIG. 7B shows a bottom view of the example cell culture container of FIG. 7A;

FIG. 7C shows a detailed cross-section of a joint between the compressible wall element and rigid base plate of the cell culture container of FIGS. 7A and 7B;

FIG. 8 shows a cross-section of an example cell culture container;

FIG. 9A shows a cross-section of an example cell culture container;

FIGS. 9B and 9C show detailed cross-sections of an attachment of the compressible wall element and the rigid base plate of the cell culture container of FIG. 9A;

FIG. 10 shows a schematic cross-section of a cell culture container showing a fluid path for gas permeability;

FIG. 11 shows an example compressible wall element of a cell culture container;

FIG. 12 shows an example compressible wall element of a cell culture container;

FIG. 13 shows an example compressible wall element of a cell culture container;

FIG. 14 shows an example supporting ring of a compressible wall element of a cell culture container; and

FIGS. 15A and 15B show example outlets of a cell culture container.

### DETAILED DESCRIPTION

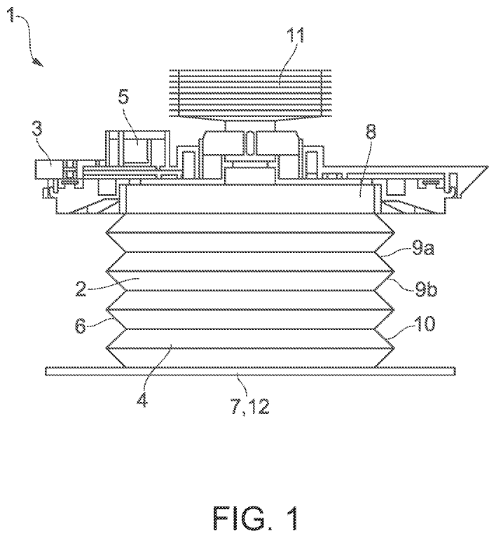

The bioreactor 1 shown in FIG. 1 includes a cell culture container 2 and an interface plate 3. During use the cell culture container 2 holds a fluid 4 in which the cell processing occurs. In particular, the fluid 4 is a cell suspension comprising a population of cells present in a liquid medium. The cells are cultured to reproduce, and may be otherwise treated, to create a cell-based therapeutic product.

The interface plate 3 is attached to a top of the cell culture container 2, for example, acting as a lid or closure. The interface plate 3 comprises at least one connector interface 5 for connecting to an external component, for example, a consumable for delivering a fluid to, or extracting a fluid from, the cell culture container 2. Accordingly, the interface plate 3 provides for adding media and other fluids to the cell culture container 2 during cell processing, and/or for removing fluid from the cell culture container 2 during processing, for example, to remove a sample or waste fluid.

The cell culture container 2 may be extendible and/or compressible. In particular, the cell culture container 2 has a compressible wall element 6, for example, a bellows wall. The cell culture container 2 has a base section 7 disposed opposite to the interface plate 3, and a compressible wall element 6 defining a sidewall of the cell culture container 2. A top part of the compressible wall element 6 is attached to the interface plate 3. The top part of the compressible wall element 6 may include a rigid ring 8 or similar for attaching to the interface plate 3. The compressible wall element 6 is compressible and/or extendible such that the base section 7 can move toward and away from the interface plate 3, changing the internal volume of the cell culture container 2. The base section 7 may be moved relative to the interface plate 3 in order to agitate or mix the fluid 4 in the cell culture container 2.

The compressible wall element 6 may be a bellows wall, having a concertina arrangement that allows the compressible wall element 6 to fold onto itself in order to compress. In particular, as illustrated the compressible wall element 6 may comprise a series of alternately arranged deformable portions 9a, 9b, specifically inwardly deformable portions 9a and outwardly deformable portions 9b. Leaf segments 10 extend between the deformable portions 9a, 9b. The leaf segments 10 are more rigid than the deformable portions 9a, 9b. The deformable portions 9a, 9b act as hinges that allow the compressible wall element 6 to collapse like a bellows or concertina, with the leaf segments 10 remaining substantially non-deformed.

The compressible wall element 6 may comprise at least one inwardly deformable portion 9a and at least one outwardly deformable portion 9b, for example, at least two inwardly deformable portions 9a and at least two outwardly deformable portions 9b. The compressible wall element 6 may comprise three, four, or more inwardly deformable portions 9a and three, four or more outwardly deformable portions 9b.

The inwardly deformable portion(s) 9a and outwardly deformable portion(s) 9b may be formed by thinned sections in the compressible wall element 6. The inwardly deformable portion(s) 9a may comprise a thinned section arranged on the outer surface of the compressible wall element 6 such that it is deformable in an inward direction. The outwardly deformable portion(s) 9b may comprise a thinned section arranged on the inner surface of the compressible wall element 6 such that it is deformable in an outwards direction.

In some examples, the compressible wall element 6 comprises a silicone, in particular, a liquid silicone rubber. In other examples, the compressible wall element 6 comprises a low density polyethylene (LDPE). In other examples, the compressible wall element 6 comprises a thermoplastic elastomer (TPE). In some examples, as described further hereinafter, the compressible wall element 6 may be coated, laminated, or otherwise treated to reduce the gas permeability of the compressible wall element 6 or to render the compressible wall element 6 impermeable to gases, particularly oxygen. In some examples, the compressible wall element 6 comprises a layer and an outer sheath, jacket, or coating. For example, the compressible wall element 6 may comprise an inner portion and a jacket over-molded onto the LDPE inner portion. The inner portion may comprise LDPE and the jacket may comprise a TPE. In another example, the compressible wall element 6 may comprise an elastomer outer, for example, a TPE outer, and a liner. For example, an LDPE liner may be blow-mounted onto the internal surface of the elastomer outer to form the liner. In another example, the liner may be an insert, for example, an LDPE insert, received within the elastomer outer but not co-molded with the elastomer outer. In such an example, it may be preferable that the liner comprises a base sheet and defines a sealed container (except for the top) to hold the cell culture.

The cell culture container 2 can, therefore, expand and contract, or be expanded and contracted, according to the material held in the cell culture container 2. In particular, the cell culture container 2 may expand as the volume of fluid 4 within the cell culture container 2 grows, and/or as additional materials are added.

As illustrated, the interface plate 3 also includes an expansion container 11, otherwise called a breathing container. The expansion container 11 allows for the cell culture container 2 to expand and contract without greatly changing the pressure in the cell culture container 2. Alternatively or additionally, the expansion container 11 may be operable, for example, by being mechanically or manually compressed or expanded, to expand or retract the compressible wall element 6 of the cell culture container 2 and thereby change a volume of the cell culture container 2. Alternatively or additionally, the expansion container 11 may be operable, for example, by being mechanically or manually compressed or expanded, to alter the pressure within the cell culture container 2.

In various examples described hereinafter, the base section 7 comprises a rigid base plate 12. The rigid base plate 12 is generally planar, i.e., flat. The rigid base plate 12 is attached to, or molded with, the compressible wall element 6, as described further hereinafter.

The rigid base plate 12 is substantially planar and thereby defines a rigid, substantially flat bottom of the cell culture container 2. A flat bottom of the cell culture container 2 may provide for improved cell culturing, in particular, mixing and control over cell culturing. The flat bottom of the cell culture container 2 helps to ensure that cells are substantially evenly spread over the cross-section of the cell culture container 2 as the cells will sink to the bottom of the cell culture container and if the base section 7 were not flat the cells would, therefore, be concentrated in a smaller volume, which may be detrimental to cell culturing. The flat bottom of the cell culture container 2 also helps to prevent fluid 4 being trapped in the cell culture container 2 when the cells are harvested or extracted at the end of the cell culturing process.

In various examples, the rigid base plate 12 comprises a thermoplastic, for example, a high density polyethylene (HDPE), or a polycarbonate (PC), or another rigid polymer. As described further hereinafter, the rigid base plate 12 may be opaque, transparent, or translucent.

In various examples as described hereinafter, the base section 7, in particular, the rigid base plate 12, has a sensor window. The sensor window is transparent or translucent and provides an optical path into the cell culture container. Accordingly, an optical sensor can transmit light into, and receive light from, a cell culture within the cell culture container.

In the various examples, the sensor window may be centrally located in the rigid base plate 12. The central position of the sensor window may ensure that the fluid 4 overlies the sensor window during mixing and agitation, so that the sensors operating through the sensor window can function.

In the illustrated examples, the cell culture container 2 is generally cylindrical, with a generally circular base section 7 and a generally cylindrical compressible wall element 6. Accordingly, an axial direction is defined between the base section 7 and the end of the compressible wall element 6 where the interface plate 3 is mounted. However, it will be appreciated that the cell culture container 2 may take an alternative form, such as having a generally triangular or square cross-sectional form.

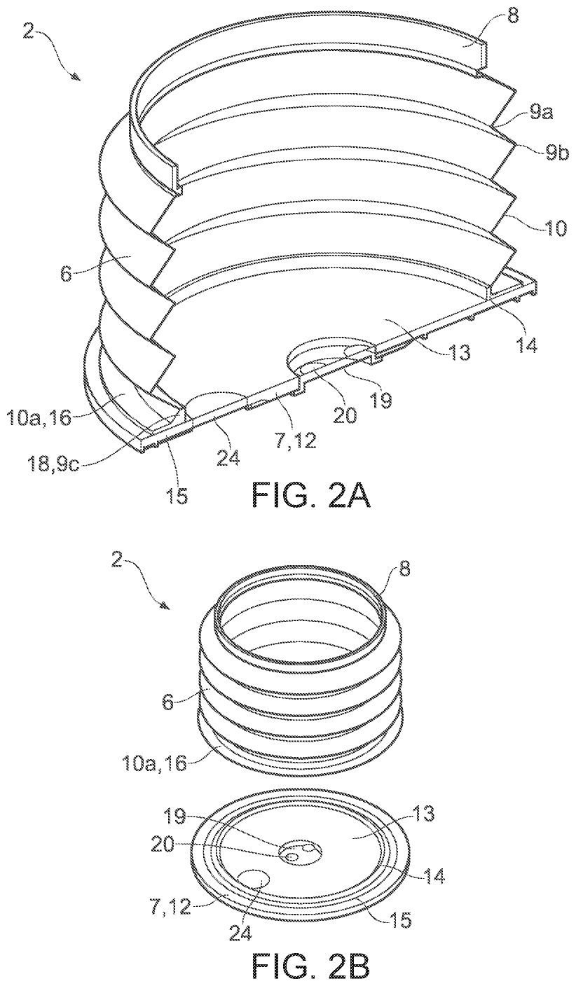

As shown in the examples of FIGS. 2A and 2B, the rigid base plate 12 comprises a planar central section 13 corresponding to the internal volume of the cell culture container 2, and a lip 14. The compressible wall element 6 is attached to the lip 14. The compressible wall element 6 may additionally or alternatively be attached to a circumferential section 15 that is outwards of the lip 14, outside of the internal volume of the cell culture container 2. In particular, the compressible wall element 6 may comprise a skirt 16 that is attached to the lip 14 and/or to the circumferential section 15. Alternatively, a bottom leaf segment 10a of the compressible wall element 6 may be attached to the lip 14 and/or to the circumferential section 15.

The compressible wall element 6, in particular, the skirt 16 or bottom leaf segment 10a, may be adhered or welded to the rigid base plate 12, in particular, the lip 14 and/or circumferential section 15. In some examples, the compressible wall element 6, in particular, the skirt 16 or bottom leaf segment 10a, is ultrasonically welded to the rigid base plate 12, in particular, the lip 14 and/or circumferential section 15. In other examples, the compressible wall element 6, in particular, the skirt 16 or bottom leaf segment 10a, is heat welded, for example, hot plate welded, to the rigid base plate 12, in particular, the lip 14 and/or circumferential section 15. The compressible wall element 6 is sealed to the rigid base plate 12.

As illustrated, the compressible wall element 6 is attached, e.g., adhered or welded, to the rigid base plate 12 such that a deformable portion 9c is positioned at or proximal to a tip 18 of the lip 14. In this illustrated example, the deformable portion 9c positioned at the tip 18 of the lip 14 is an inwardly deformable portion.

In this example, the base section 7, in particular, the rigid base plate 12, comprises an opaque HDPE material. The rigid base plate 12 may be molded, for example, injection molded.

As shown in FIGS. 2A and 2B, the base section 7 also includes a sensor window 19. In this example, the sensor window 19 comprises a transparent or translucent window. For example, the sensor window 19 may comprise a polycarbonate window attached to, or molded into, an opening in the rigid base plate 12. The sensor window 19 may be an insert in the opening in the rigid base plate 12.

One or more sensor elements 20 may be attached to, or molded into, the sensor window 19. The sensor element 20 may be an optical dot for use with an optical sensor, for example, to detect a dissolved oxygen content of the fluid in the cell culture container 2 during use.

In other examples, the rigid base plate 12 may comprise a transparent or translucent material, for example, polycarbonate (PC), and the sensor window 19 may be defined as a part of the planar central section 13.

As schematically illustrated, the rigid base plate 12 may additionally comprise a valve 24 for extraction of fluid from the cell culture container 2, for example, for harvesting cells from the cell culture container 2 at the end of a cell culturing process.

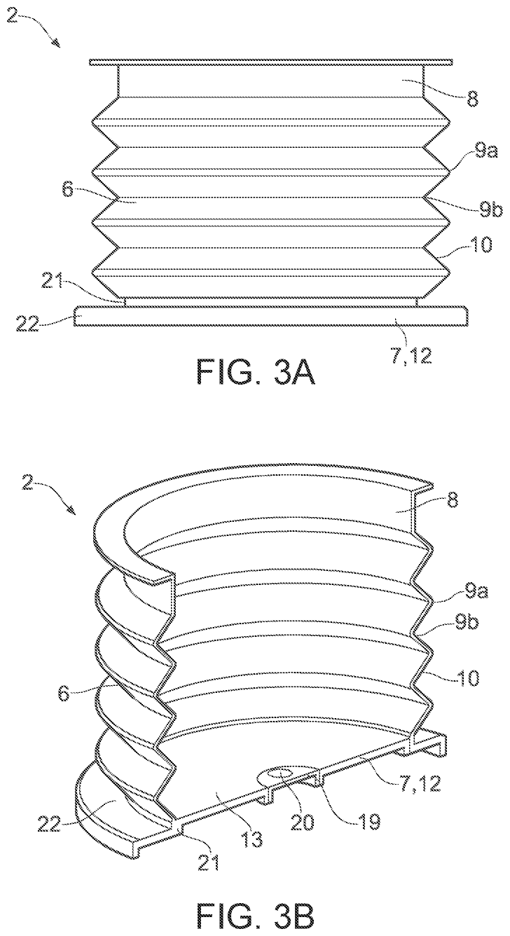

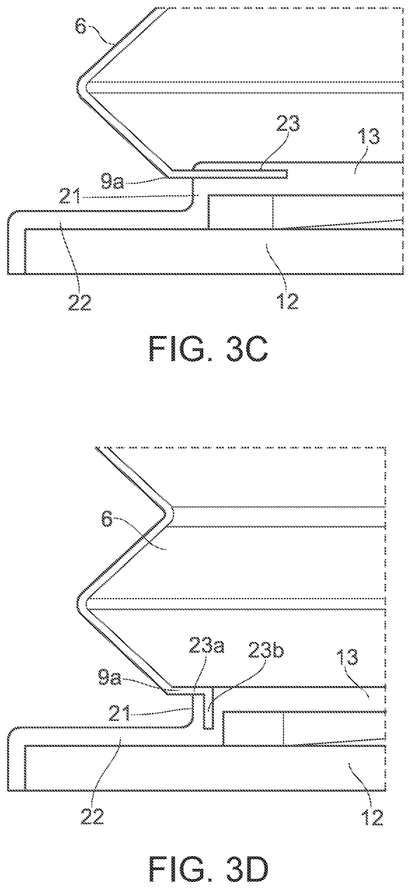

In the examples shown in FIGS. 3A to 3D, the rigid base plate 12 comprises a planar central section 13 corresponding to the internal volume of the cell culture container 2. In this example, the rigid base plate 12 comprises a shoulder 21 such that a circumferential portion 22 is stepped with respect to the planar central section 13. As illustrated, particularly in FIGS. 3C and 3D, the compressible wall element 6 is molded into the rigid base plate 12 at the shoulder 21. In the illustrated example, the shoulder 21 provides an outer circumferential surface to which the compressible wall element 6 is attached. However, it will be appreciated that the shoulder 21 may extend in the opposite direction to provide a recess having an internal circumferential surface to which the compressible wall element 6 is attached.

In the examples of FIGS. 3A to 3D, a portion of the skirt 23 at the end of the compressible wall element 6 is molded into, in particular, embedded in, the rigid base plate 12 and so provides a sealed connection between the compressible wall element 6 and the rigid base plate 12.

In the example of FIG. 3C, a skirt 23 of the compressible wall element 6 is molded into the rigid base plate 12 at the shoulder 21. The skirt 23 extends from an inwardly deformable portion 9a and the skirt extends radially inward toward the planar central section 13. The skirt 23 may be molded into the rigid base plate 12 by a two-stage molding process, in particular, a two-stage injection molding process.

In the example of FIG. 3D, the skirt 23 of the compressible wall element 6 is molded into the rigid base plate 12 at the shoulder 21. The skirt 23 has first part 23a that extends from an inwardly deformable portion 9a in a radially inward direction toward the planar central section 13. The first part 23a overlies a part of the planar central section 13 and may or may not be molded into the rigid base plate 12. The skirt 23 also has a second part 23b that extends into the rigid base plate 12 in a perpendicular direction to the first part 23a, in a generally axial direction. The second part 23b of the skirt 23 is molded into the rigid base plate 12. The second part 23b of the skirt 23 may be molded into the rigid base plate 12 by a two-stage molding process, in particular, a two-stage injection molding process.

In the examples of FIGS. 3A to 3D, the inwardly deformable portion 9a is positioned at the shoulder 21, and so the compressible wall element 6 can be folded against the rigid base plate 12, in particular, the planar central section 13.

In this example, the base section 7, in particular, the rigid base plate 12, comprises an opaque HDPE material.

As shown in FIG. 3B, the base section 7 also includes a sensor window 19. In this example, the sensor window 19 comprises a transparent or translucent window. For example, the sensor window 19 may comprise a polycarbonate window attached to, or molded into, an opening in the rigid base plate 12. The sensor window 19 may be an insert in the opening in the rigid base plate 12.

One or more sensor elements 20 may be attached to, or molded into, the sensor window. The sensor element 20 may be an optical dot for use with an optical sensor, for example, to detect a dissolved oxygen content of the fluid in the cell culture container 2 during use.

In other examples, the rigid base plate 12 may comprise a transparent or translucent material, for example, a polycarbonate, and the sensor window 19 may be defined as a part of the planar central section 13.

Similarly to the examples of FIGS. 2A and 2B, the rigid base plate 12 may additionally comprise a valve (24, see FIG. 2A) for extraction of fluid from the cell culture container 2, for example, for harvesting cells from the cell culture container 2 at the end of a cell culturing process.

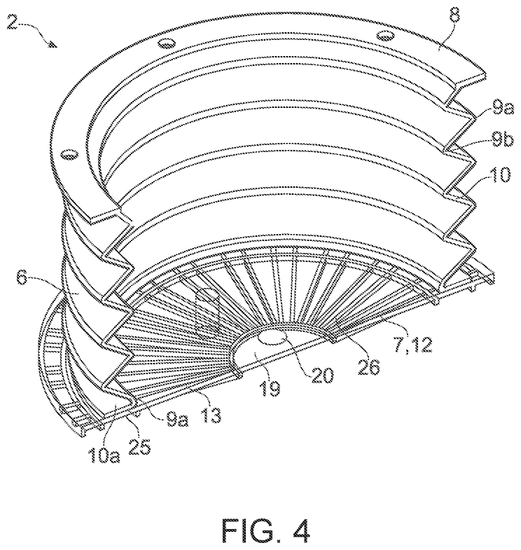

In the example of FIG. 4, the rigid base plate 12 comprises a planar central section 13 corresponding to the internal volume of the cell culture container 2. The rigid base plate 12 also comprises a circumferential section 25 radially outward of the planar central section 13. A bottom leaf segment 10a of the compressible wall element 6 is attached to the circumferential section 25. For example, the bottom leaf segment 10a is adhered or welded to the circumferential section 25. The bottom leaf segment 10a may be hot plate welded or ultrasonically welded to the circumferential section 25. The compressible wall element 6 is sealed to the rigid base plate 12.

As illustrated, the bottom leaf segment 10a is attached to the circumferential section 25 such that an inwardly deformable portion 9a of the compressible wall element 6 is arranged at, or immediately adjacent to, the rigid base plate 12.

In this example, the base section 7, in particular, the rigid base plate 12, comprises a transparent polycarbonate (PC) material. The rigid base plate 12 comprises a plurality of strengthening ribs 26 molded into a surface of the rigid base plate 12 opposite to the compressible wall element 6 to improve the strength and rigidity of the rigid base plate 12 and reduce the risk of shattering.

As shown in FIG. 4, the base section 7 also includes a sensor window 19. In this example, the sensor window 19 comprises a planar portion in the rigid base plate 12, where any strengthening ribs 26 do not extend.

One or more sensor elements 20 may be attached to, or molded into, the rigid base plate 12 at the sensor window 19. The sensor element 20 may be an optical dot for use with an optical sensor, for example, to detect a dissolved oxygen content of the fluid in the cell culture container 2 during use.

Alternatively, similar to the examples of FIGS. 2A to 3D, the rigid base plate 12 may comprise an opaque material such as an opaque HDPE, and the sensor window 19 may comprise an integrally molded, attached, or inserted transparent material, such as a polycarbonate insert.

Similarly to the examples of FIGS. 2A and 2B, the rigid base plate 12 may additionally comprise a valve (24, see FIG. 2A) for extraction of fluid from the cell culture container 2, for example, for harvesting cells from the cell culture container 2 at the end of a cell culturing process.

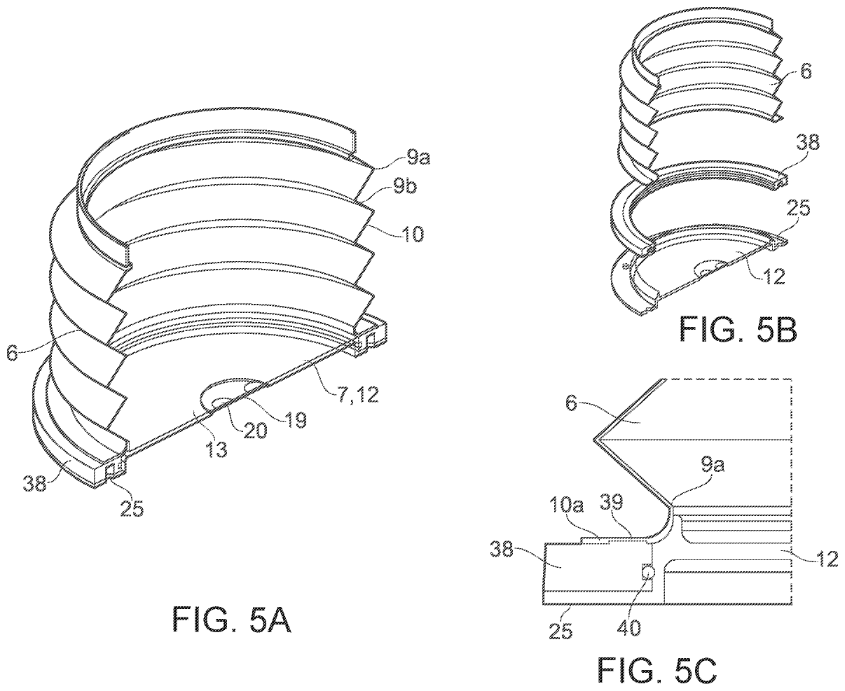

In the example of FIGS. 5A to 5C, the cell culture container 2 comprises a rigid base plate 12 with a planar central section 13 corresponding to the internal volume of the cell culture container 2. The cell culture container 2 also has a compressible wall element 6, which in this example, is attached to the rigid base plate 12 by a ring 38. The rigid base plate 12 comprises a circumferential section 25 radially outward of the planar central section 13. A bottom leaf segment 10a of the compressible wall element 6 is attached to the ring 38, for example, by adhesive or by a weld such as an ultrasonic weld or a hot plate weld 39. The compressible wall element 6 is sealed to the ring 38. The ring 38 is attachable to the circumferential section 25 of the rigid base plate 12, for example, by one or more fasteners. A seal, for example, an O-ring 40, may be provided between the ring 38 and the rigid base plate 12 to provide a sealed attachment of the ring 38 to the rigid base plate 12.

As illustrated in FIG. 5C, the bottom leaf segment 10a is attached to the ring 38 such that an inwardly deformable portion 9a of the compressible wall element 6 is arranged at, or immediately adjacent to, the rigid base plate 12.

In this example, the base section 7, in particular, the rigid base plate 12, comprises a transparent polycarbonate (PC) material.

As shown in FIG. 4, the base section 7 also includes a sensor window 19. One or more sensor elements 20 may be attached to, or molded into, the rigid base plate 12 at the sensor window 19. The sensor element 20 may be an optical dot for use with an optical sensor, for example, to detect a dissolved oxygen content of the fluid in the cell culture container 2 during use.

Alternatively, similar to the examples of FIGS. 2A to 3D, the rigid base plate 12 may comprise an opaque material such as an opaque HDPE, and the sensor window 19 may comprise an integrally molded, attached, or inserted transparent material, such as a polycarbonate insert.

Similarly to the examples of FIGS. 2A and 2B, the rigid base plate 12 may additionally comprise a valve (24, see FIG. 2A) for extraction of fluid from the cell culture container 2, for example, for harvesting cells from the cell culture container 2 at the end of a cell culturing process.

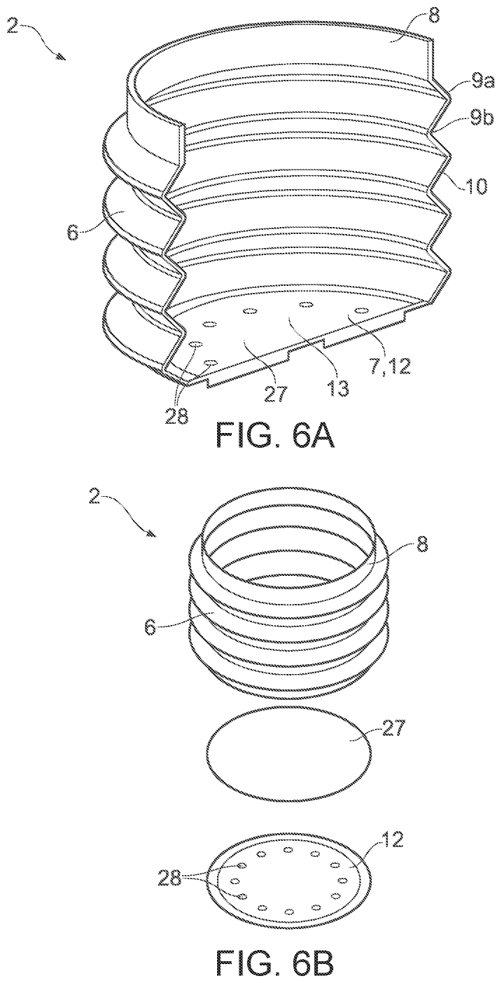

In the example of FIGS. 6A and 6B, the cell culture container 2 comprises a compressible wall element 6 and a rigid base plate 12 that may be attached to each other in any of the ways described with reference to FIGS. 2A to 5C. In this example, as shown more clearly in FIG. 6B, the cell culture container 2 further comprises a base sheet 27. The base sheet 27 extends across the rigid base plate 12 within the cell culture container 2.

In some examples, the base sheet 27 is attached, in particular, sealingly attached, to the compressible wall element 6. The internal volume of the cell culture container 2 is thereby sealed between the compressible wall element 6 and the base sheet 27, and the fluid does not contact the rigid base plate 12. In this example, the base sheet 27 is sealingly attached to the compressible wall element 6, for example, by welding such as hot plate welding or ultrasonic welding.

Additionally or alternatively, the base sheet 27 is attached, in particular, sealingly attached, to the rigid base plate 12. The base sheet 27 can be attached to the rigid base plate 12 around a circumference of the base sheet 27, at or adjacent to the joint between the compressible wall element 6 and the rigid base plate 12. The internal volume of the cell culture container 2 is thereby sealed between the compressible wall element 6 and the base sheet 27. In this example, the base sheet 27 is sealingly attached to the rigid base plate 12, for example, by welding such as hot plate welding or ultrasonic welding.

In some examples, the base sheet 27 may gas permeable. In particular, the base sheet 27 may be oxygen permeable. The base sheet 27 may comprise silicone, in particular, liquid silicone rubber.

The rigid base plate 12 comprises one or more openings, in particular, holes 28. Accordingly, the base sheet 27 is exposed to atmosphere through the holes 28 and gas, for example, oxygen, can permeate through the base sheet 27.

In this example, the compressible wall element 6 may be gas permeable, in particular, oxygen permeable, or may gas impermeable, in particular, oxygen impermeable. In some examples, the compressible wall element 6 may be coated or laminated to render the compressible wall element 6 gas impermeable, in particular, oxygen impermeable. In some examples, the compressible wall element 6 comprises an inner silicone layer and an outer LDPE sheath or coating. The inner silicone layer may be an internal liner of the outer LDPE sheath.

As with previous examples, the rigid base plate 12 may also include a sensor window. The base sheet 27 may be transparent or translucent. The sensor window may comprise a transparent or translucent window attached to, or molded into, an opening in the rigid base plate 12. The sensor window may be an insert in the opening in the rigid base plate 12. The rigid base plate 12 may be transparent and the sensor window may be a portion of the rigid base plate 12.

One or more sensor elements may be attached to, or molded into, the rigid base plate 12 at the sensor window. The sensor element may be an optical dot for use with an optical sensor, for example, to detect a dissolved oxygen content of the fluid in the cell culture container 2 during use.

Similarly to the examples of FIGS. 2A and 2B, the rigid base plate 12 may additionally comprise a valve (24, see FIG. 2A) for extraction of fluid from the cell culture container 2, for example, for harvesting cells from the cell culture container 2 at the end of a cell culturing process.

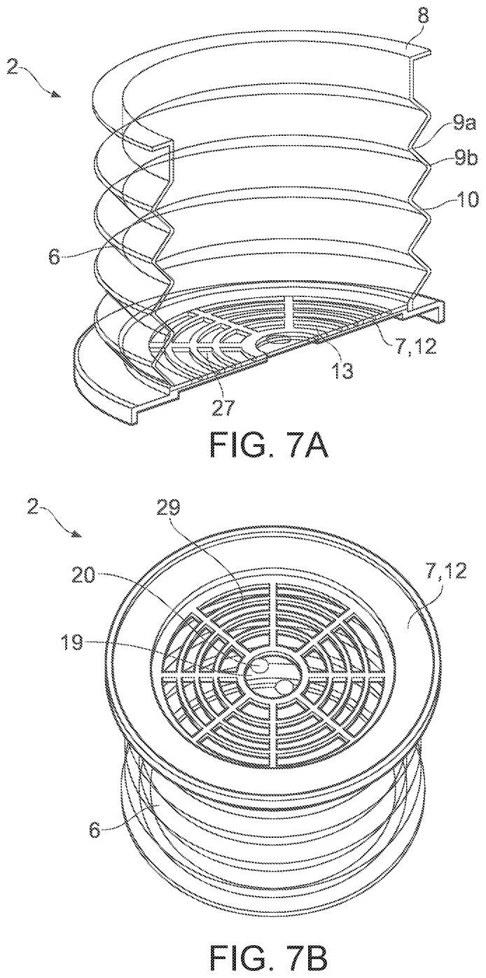

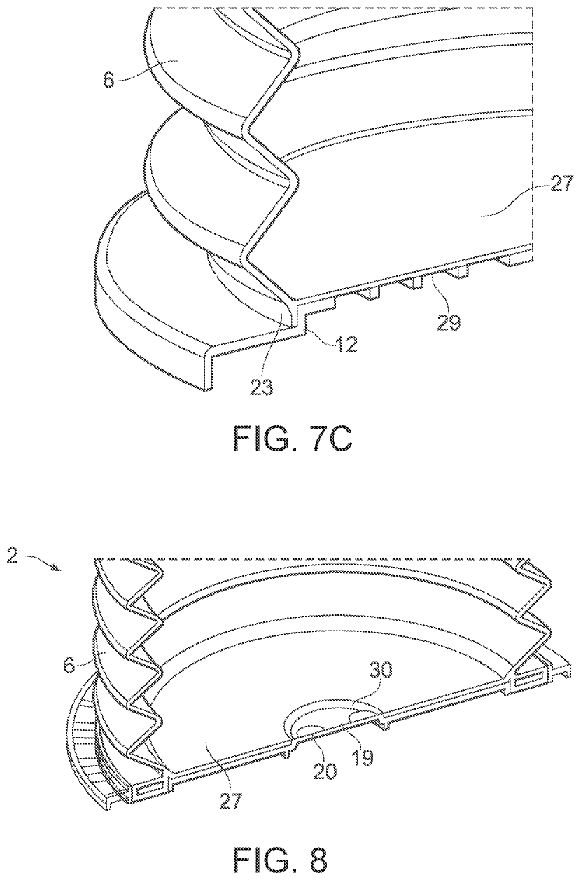

In the examples of FIGS. 7A to 7C, the cell culture container 2 comprises a compressible wall element 6 and a rigid base plate 12 that may be attached to each other in any of the ways described with reference to FIGS. 2A to 5C. In this example, as shown most clearly in FIGS. 7A and 7C, the cell culture container 2 further comprises a base sheet 27. The base sheet 27 extends across the rigid base plate 12 within the cell culture container 2.

In this example, the base sheet 27 is a part of the compressible wall element 6. In particular, the base sheet 27 is formed as part of the same molding as the compressible wall element 6, for example, by blow molding. As shown in FIG. 7C, the compressible wall element 6 may comprise a skirt 23, similar to as described with reference to FIGS. 3A to 3D, that is attached to the rigid base plate 12. In other examples, the compressible wall element 6 may be attached, for example, adhered or welded to, the rigid base plate 12 similar to as described with reference to FIGS. 2A, 2B, 4, and 5A to 5C.

In the example of FIGS. 7A to 7C, the internal volume of the cell culture container 2 is thereby defined only within the compressible wall element 6 and there is no sealed joint between the compressible wall element 6 and the rigid base plate 12. Therefore, the fluid does not contact the rigid base plate 12.

In some examples, a portion of the base sheet 27 is attached to the rigid base plate 12, for example, by adhesive or welding, in particular, spot welding. The base sheet 27 can be attached to the rigid base plate 12 around a circumference of the base sheet 27.

In some examples, the base sheet 27 may gas permeable. In particular, the base sheet 27 may be oxygen permeable. The base sheet 27 may comprise silicone, such as liquid silicone rubber. The base sheet 27 is made from the same material as the compressible wall element 6. All or a part of the compressible wall element 6 may be coated or laminated to render it gas impermeable, in particular, oxygen impermeable. In some examples, the compressible wall element 6 comprises an inner silicone layer and an outer LDPE sheath or coating. The inner silicone layer may be a liner of the LDPE sheath.

In this example, as shown in FIGS. 7B and 7C, the rigid base plate 12 comprises one or more openings 29. Accordingly, the base sheet 27 is exposed to atmosphere through the openings 29 and gas, for example, oxygen, can permeate through the base sheet 27.

As with previous examples, the rigid base plate 12 may also include a sensor window 19, as shown in FIG. 7B. The base sheet 27 may be transparent or translucent. The sensor window 19 may comprise a transparent or translucent window attached to, or molded into, an opening in the rigid base plate 12. The sensor window 19 may be an insert in the opening in the rigid base plate 12. The rigid base plate 12 may be transparent and the sensor window 19 may be a portion of the rigid base plate 12.

One or more sensor elements 20 may be attached to, or molded into, the rigid base plate 12 at the sensor window 19. The sensor element 20 may be an optical dot for use with an optical sensor, for example, to detect a dissolved oxygen content of the fluid in the cell culture container 2 during use.

Similarly to the examples of FIGS. 2A and 2B, the rigid base plate 12 may additionally comprise a valve (24, see FIG. 2A) for extraction of fluid from the cell culture container 2, for example, for harvesting cells from the cell culture container 2 at the end of a cell culturing process.

In the example of FIG. 8, similar to the examples of FIGS. 6A to 7C, the compressible wall element 6 includes a base sheet 27. In this example, the base sheet 27 is opaque. As shown, the base sheet 27 includes an opening 30 aligned with a sensor window 19 in the rigid base plate 12. The base sheet 27 is sealingly attached to the rigid base plate 12 about the opening 30. For example, the base sheet 27 is welded or adhered to the rigid base plate 12 about the opening 30.

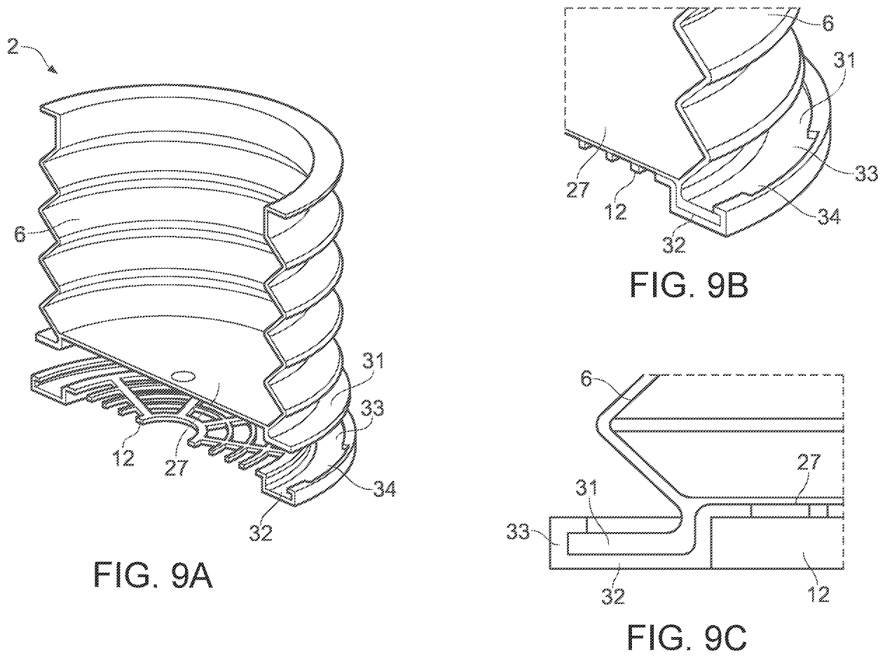

In the example of FIGS. 9A to 9C, the compressible wall element 6 is attached to the rigid base plate 12 by clipping. The rigid base plate 12 and compressible wall element 6 may be as described with the examples of FIGS. 6A to 8. In particular, as illustrated, in this example, the compressible wall element 6 comprises an integral base sheet 27 so that the internal volume of the cell culture container 2 is defined within the compressible wall element 6.

The compressible wall element 6 comprises a flange 31 extending generally radially. The flange 31 may be flexible or deformable, and/or may comprise an increased thickness so as to have a greater stiffness. The flange 31 is received in a groove 33 formed in a circumferential portion 32 of the rigid base plate 12. The groove 33 is shaped to receive the flange 31 of the compressible wall element 6 such that the flange 31 is retained in the groove 33. The groove 33 may include one or more scalloped sections 34 to ease insertion of the flange 31 into the groove 33. Accordingly, the compressible wall element 6 can be clipped onto the rigid base plate 12 by clipping the flange 31 into the groove 33.

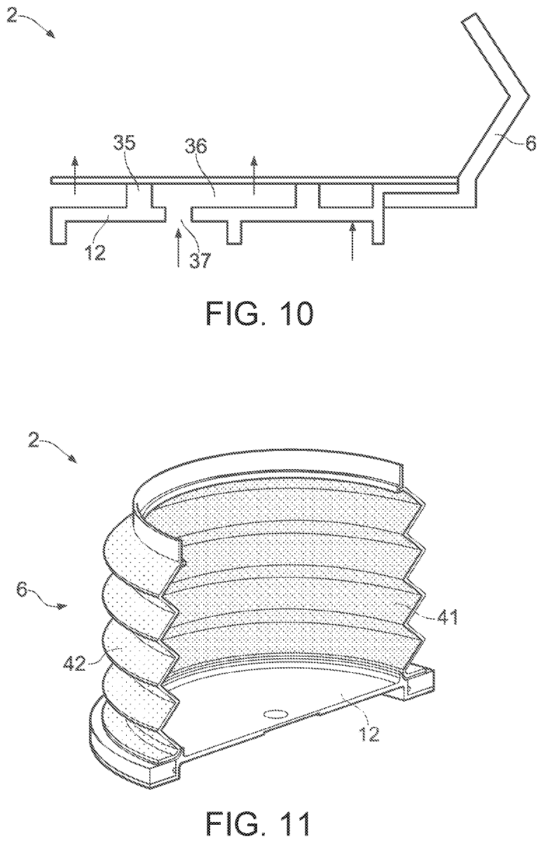

FIG. 10 illustrates an example cell culture container 2 having a base sheet 27, for example, as described with reference to FIGS. 6A to 9C. The base sheet 27 may be integral with the compressible wall element 6 or separate, as illustrated in FIG. 10. In the illustrated example, the base sheet 27 is sealed to the rigid base plate 12 within the internal volume of the cell culture container 2.

In this example, the base sheet 27 is gas permeable, in particular, oxygen permeable. The rigid base plate 12 comprises one or more openings 29 and one or more spacing ribs 35 extending from the rigid base plate 12 toward the internal volume of the cell culture container 2, in particular, toward the base sheet 27. In this example, the spacing ribs 35 are arranged to space the base sheet 27 from the rigid base plate 12 to create fluid channels 36, 37 for gas circulation, in particular, air circulation. Accordingly, air can reach the underside of the base sheet 27 and permeate into the cell culture container 2 during use.

FIGS. 11 to 13 illustrate different compressible wall elements 6 that may be used with any of the cell culture containers 2 described with reference to FIGS. 2A to 10. In particular, each of FIGS. 11 to 13 illustrates a compressible wall element 6 attached to a rigid base plate 12 to form the cell culture container 2.

In the example of FIG. 11, the compressible wall element comprises an inner portion 41 and a jacket 42. In this example, the jacket 42 is over-molded onto the inner portion 41, so that they are integrally formed. In some examples, the inner portion 41 comprises an LDPE and the jacket 42 comprises a thermoplastic elastomer (TPE).

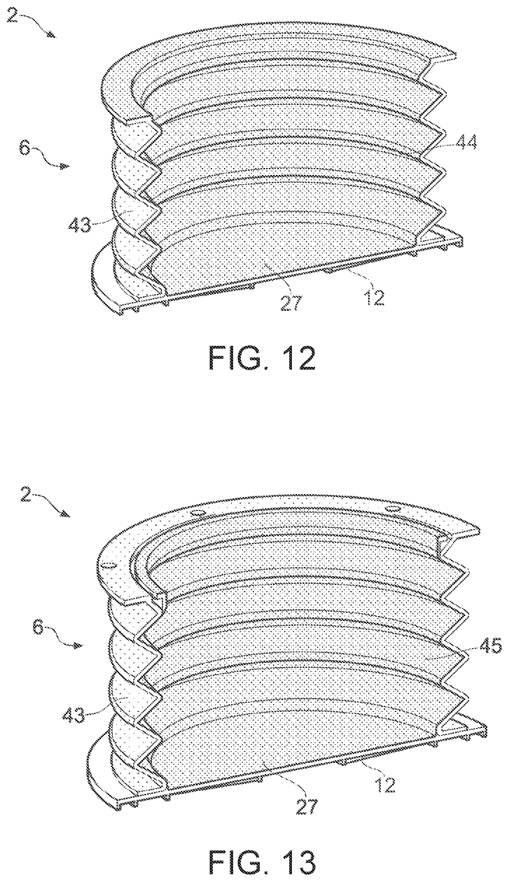

In the example of FIG. 12 the compressible wall element 6 comprises an elastomer outer 43, for example, a TPE outer, and a liner 44. For example, an LDPE liner 44 may be blow-mounted onto the internal surface of the elastomer outer 43 to form the liner 44. As shown, the liner 44 includes a base sheet 27 as previously described.

In the example of FIG. 13, the compressible wall element comprises an elastomer outer 43, for example, a TPE outer, and an insert 45. The insert 45 may comprise LDPE. The insert 45 is received within the elastomer outer 43 but not co-molded with the elastomer outer 43. As illustrated, the insert 45 comprises a base sheet 27 as previously described.

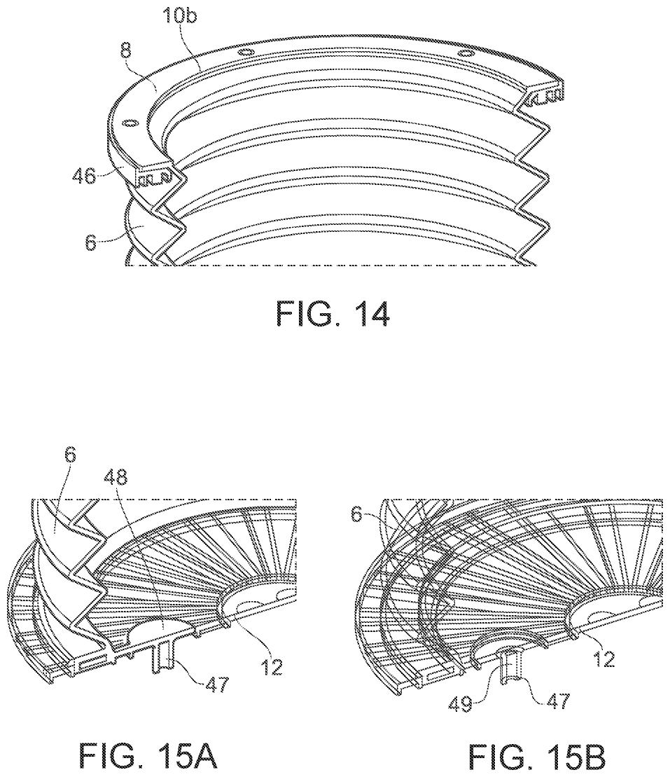

FIG. 14 illustrates a cell culture container with a support ring 46 provided at the top end of the compressible wall element 6. The support ring 46 is attached to a top-most leaf segment 10b of the compressible wall element 6, for example, by adhesive, welding, or fasteners. The support ring 46 is rigid and may be engaged by another part of the bioreactor 1 illustrated in FIG. 1, in particular, the interface plate 3. It will be appreciated that the support ring 46 may be provided on the example cell culture container 2 of any other example described herein.

FIGS. 15A and 15B illustrate examples of providing an outlet 47 for extraction of fluid from the cell culture container, for example, for harvesting cells from the cell culture container at the end of a cell culturing process. The outlet 47 is formed in the rigid base plate 12, so is formed at a lower end of the cell culture container and gravity can assist in harvesting cells through the outlet 47. The outlet 47 may comprise a valve or openable closure.

In the example of FIG. 15A, the outlet 47 is formed in an opening in the rigid base plate 12, within an insert 48 that seals the opening. The insert 48 may be a thermoplastic elastomer. As illustrated, the insert 48 may be a part of the compressible wall element 6 that extends from the compressible wall element 6 in a direction across the rigid base plate 12 to the opening. The insert 48 may be adhered or otherwise attached to the rigid base plate 12 to provide a seal between the insert 48 and the rigid base plate 12.

In the example of FIG. 15B, the outlet 47 is formed as a part of the rigid base plate 12, in particular, in a protrusion 49 of the rigid base plate 12.

Throughout the description and claims of this specification, the words “comprise” and “contain” and variations of them mean “including but not limited to,” and they are not intended to (and do not) exclude other components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the disclosure are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The disclosure is not restricted to the details of any foregoing embodiments. The disclosure extends to any novel one, or any novel combination, of the features disclosed in this specification (including any accompanying claims, abstract and drawings), or to any novel one, or any novel combination, of the steps of any method or process so disclosed.

1. A cell culture container comprising:
a base section including a substantially planar rigid base plate; and
a compressible wall element extending from the base section in an axial direction and defining an internal volume of the cell culture container, the compressible wall element being compressible in the axial direction.

2. The cell culture container of claim 1, wherein the compressible wall element is attached to the rigid base plate by a connection chosen from a weld, a clamp, a clip, or an adhering connection.

3. (canceled)

4. The cell culture container of claim 1, wherein the compressible wall element is integrally molded with the rigid base plate as an over-mold onto a part of the compressible wall element.

5. The cell culture container of claim 1, wherein the compressible wall element comprises a deformable portion disposed at a position chosen from at a joint between the compressible wall element and the rigid base plate or immediately adjacent to the joint between the compressible wall element and the rigid base plate.

6. The cell culture container of claim 1, further comprising a base sheet extending over the rigid base plate within the internal volume of the cell culture container.

7. The cell culture container of claim 6, wherein the base sheet extends from the compressible wall element and is integrally molded with the compressible wall element.

8. The cell culture container of claim 6, wherein the base sheet is oxygen permeable.

9. The cell culture container of claim 8, wherein the rigid base plate comprises one or more gas permeable openings.

10. The cell culture container of claim 8, wherein the rigid base plate comprises one or more spacers adapted to space the base sheet from the rigid base plate.

11. The cell culture container of claim 1, wherein the compressible wall element comprises an inwardly deformable portion, an outwardly deformable portion, and a leaf portion extending between the inwardly deformable portion and the outwardly deformable portion, and wherein the inwardly deformable portion and the outwardly deformable portion are adapted to deform to cause the compressible wall element to compress.

12. The cell culture container of claim 11, wherein a portion chosen from the inwardly deformable portion or the outwardly deformable portion is disposed at a position chosen from at a joint between the compressible wall element and the rigid base plate or immediately adjacent to the joint between the compressible wall element and the rigid base plate.

13. The cell culture container of claim 1, wherein the rigid base plate comprises a sensor window chosen from a transparent sensor window or a translucent sensor window.

14. The cell culture container of claim 1, wherein the compressible wall element comprises a material chosen from a silicone, a low density polyethylene, or a thermoplastic elastomer.

15. The cell culture container of claim 1, wherein the compressible wall element comprises an outer portion and an element chosen from a liner or an insert.

16. The cell culture container of claim 1, wherein the compressible wall element comprises an inner portion and a jacket.

17. The cell culture container of claim 1, wherein at least a part of the compressible wall element comprises a gas impermeable coating.

18. The cell culture container of claim 1, wherein the rigid base plate comprises a material chosen from a high density polyethylene or a polycarbonate.

19. (canceled)

20. A bioreactor for a cell culturing process, the bioreactor comprising:
a cell culture container including
a base section including a substantially planar rigid base plate, and
a compressible wall element extending from the base section in an axial direction and defining an internal volume of the cell culture container, the compressible wall element being compressible in the axial direction; and
an interface plate attachable to the compressible wall element opposite to the base section to close the cell culture container.

21. (canceled)

22. The bioreactor of claim 20, wherein the interface plate comprises a connector interface.

23. A cell processing system, comprising:
a bioreactor comprising a cell culture container including a base section including a substantially planar rigid base plate, and a compressible wall element extending from the base section in an axial direction and defining an internal volume of the cell culture container, the compressible wall element being compressible in the axial direction; and
an agitator configured to move the base section to agitate a fluid in the cell culture container.

24. (canceled)
